(19) United States
(12) Patent Application Publication
(10) Pub. No.: US 2015/0147240 A1
(43) Pub. Date: May 28, 2015

(54) Led Lamp Having Photocatalyst Agents

(71) Applicants: Liann-Be Chang, Taoyuan County (TW); Fu-Cheng Chuang, Taoyuan County (TW); Shih-Huan Huang, Taoyuan County (TW)

(72) Inventors: Liann-Be Chang, Taoyuan County (TW); Fu-Cheng Chuang, Taoyuan County (TW); Shih-Huan Huang, Taoyuan County (TW)

(21) Appl. No.: 14/559,987

(22) Filed: Dec. 4, 2014

Related U.S. Application Data
(63) Continuation-in-part of application No. 12/762,635, filed on Apr. 19, 2010.
Application No. 12/762,635 is a continuation-in-part of application No. 11/852,461, filed on Sep. 10, 2007, now abandoned.

(51) Int. Cl. A61L 9/18

(52) U.S. Cl. CPC A61L 9/18
USPC 422/186

(57) ABSTRACT

An LED lamp is provided with at least one LED each including an epoxy lens, cathode and anode leads; a reflective cup, a LED chip, and a wire bond; a housing for accommodating a circuit board, the at least one LED mounted on the circuit board, and a fan; openings on the housing; groups of photocatalyst agents coated on areas lit by the LED chip wherein the photocatalyst agents are TiO₂ having a particle size in the range of 50 and 300 nano meters; and layers of fluorescent substance coated on areas lit by the LED chip wherein emitted light impinges the layers of fluorescent substance to generate fluorescent light, and wherein white light is generated when emitted light mixes with the fluorescent light. The LED lamp has the functions of disinfection, deodorization, and mildew proofing.

### CROSS-REFERENCE TO RELATED APPLICATION

This application is a continuation in part of U.S. application Ser. No. 12/762,635, filed Apr. 19, 2010, entitled “LED Structure”. The entire teachings of the above application are incorporated herein by reference.

### BACKGROUND OF THE INVENTION

1. Field of the Invention

The invention relates to LED (light-emitting diode) lamps, and more particularly to an LED lamp having photocatalyst agents which can be reacted to achieve functions including disinfection, deodorization, and mildew proofing.

2. Description of the Prior Art

A photocatalyst agent is a catalyst that is related to light and transformed by light energy, such transformation is a sort of chemistry. As a matter of fact, titanium dioxide (TiO₂) is a catalyst that achieves the transformation. Titanium dioxide itself is an activator with the characteristics of hyper oxidation and stability. A photocatalyst agent such as titanium dioxide is powerful enough to resolve almost all substances yet is very safe. Moreover, titanium dioxide is superior to ammonia, NH₃, etc. for water treatment. A photocatalyst that promotes photosynthesis similar to a plant's photosynthesis is produced after titanium dioxide absorbs ultraviolet ray from sunlight or a conventional LED. The oxidation from the photocatalyst easily reduces germs in air by 99.997%.

The photocatalyst agents of the invention perform three functions including disinfection, deodorization, and mildew proofing, which are described as below:

Disinfection: Germs in water or in air that contacts a surface treated by the photocatalyst agents and activated by ultraviolet rays so that air can be easily removed due to oxidation.

Deodorization: Sources of odor include ammonia, hydrogen sulfide, methyl mercaptide, formaldehyde, etc. Titanium dioxide has the capability to oxidize the source that is greater than ammonia, and a capability of adsorption that is better than activated carbon related to reducing germs. While such substances exist under the circumstance of general light sources, titanium dioxide is capable of easily removing hydrogen sulfide, sulfur dioxide, etc. of a cigarette. Materials such as hydrogen sulfide, sulfur dioxide, and the like are known carcinogens.

Mildew proofing: Mildew is formed on a surface by mold. The photocatalyst agents can solve the problem very easily, such as by using the agents as a coating on a cover or wall to prevent mold. In this manner food can be protected for a longer duration to avoid illness, and the wall is kept clean to avoid dust.

According to aforesaid, the photocatalyst agents have the functions of disinfection, deodorization, and mildew proofing. But so far in the art it has not been disclosed in conventional LEDs. The total contact surface area of a conventional LED is smaller than an improved LED lamp as the proposed LED assembly. While the inventor developed an improved LED lamp to solve the shortcomings of the prior art, continuous improvements of the art are always sought by the inventor.

### SUMMARY OF THE INVENTION

Conventionally, the photocatalyst agents are only applied to typical light bulbs. The total contact surface area of a conventional LED is smaller than an improved LED lamp such as an LED assembly, which can have a plurality of LEDs. As the general purpose of the conventional LED is solid state lighting, the surface area of a conventional LED contacting air is very limited, and the corresponding oxidation capability from the conventional LED is limited and less than the desired amount of disinfection, deodorization, and mildew proofing.

A main object of the invention is to provide an LED lamp having photocatalyst agents, not only for solid state light but also for disinfection, deodorization, and mildew proofing functions. Moreover, by increasing the total contact , surface area of photocatalytic nanoparticles to increase the surface being in contact with air, the disinfection, deodorization, and mildew proofing functions can be strengthened.

The LED lamp comprises

at least one LED each comprising an epoxy lens, a cathode lead extending from a bottom of the epoxy lens, an anode lead extending from the bottom of the epoxy lens, a reflective cup disposed in the epoxy lens, a LED chip disposed in the epoxy lens and connected to the reflective cup for electrically connecting to the cathode lead, and a wire bond disposed in the epoxy lens and electrically interconnected the LED chip and the anode lead wherein light emitted by the LED chip has a wavelength less than 400 nm; a housing for accommodating a circuit board, the at least one LED mounted on the circuit board and electrically connected to the circuit board, and a fan electrically connected to the circuit board; at least one opening formed on the housing for communicating with inside of the housing; a plurality of groups of photocatalyst agents coated on areas lit by the LED chip of the at least one LED wherein the groups of photocatalyst agents are separated from the LED chip of the at least one LED, and the photocatalyst agents are TiO₂ having a particle size in the range of 50 and 300 nano meters; and a plurality of layers of fluorescent substance coated on transparent areas lit by the LED chip of the at least one LED wherein light emitted by the at least on LED is configured to impinge the layers of fluorescent substance to generate fluorescent light, and wherein white light is generated when light emitted by the at least one LED mixes with the fluorescent light.

The activated fan of the invention can increase ventilation of the space. And in turn, more photocatalyst agents are in contact with air so as to kill more microorganisms in the air. The LED lamp having photocatalyst agents can be installed in an inlet of an air conditioner. It is understood that outlet of an air conditioner has a lower temperature. Dew may be formed at the outlet and it can adversely affect activity of nanoparticles of the photocatalyst agent. To the contrast, inlet of the air conditioner is drier with less dew being formed. It is preferred to install the LED lamp and an additional fan at the inlet of the air conditioner so as to increase ventilation. Therefore, the photocatalyst agents can perform the functions of disinfection, deodorization, and mildew proofing.

The fluorescent substance can be coated on the housing or the diffusing board. The fluorescent substance can be used again after replacing a malfunctioned LED with a new one. This can reduce the consumption of fluorescent substance.

Size of an LED is smaller and is more convenient to install than conventional light bulbs. Comparing the invention to a conventional LED of the same size, the invention can increase the total surface area contacting air, so that the functions of disinfection, deodorization, and mildew proofing can be effectively performed.

The above and other objects, features and advantages of the invention will become apparent from the following detailed description taken with the accompanying drawings.

### BRIEF DESCRIPTION OF THE DRAWINGS

FIG. 1 is a perspective view of an LED of a preferred embodiment of the invention;

FIG. 2 is a detailed view of the area in a circle of FIG. 1;

FIG. 3 is a side elevation in part section of a cylindrical light source incorporating LEDs of the invention as a first preferred embodiment;

FIG. 4A is a sectional view taken along line A-A of FIG. 3;

FIG. 4B is a detailed view of the area in a circle of FIG. 4A;

FIG. 5 is a longitudinal sectional view of the light sources mounted in a lamp casing of an LED lamp;

FIG. 6 is a longitudinal sectional view of a bulb shaped LED lamp incorporating the LEDs of the invention as a second preferred embodiment;

FIG. 7 is a schematic view of chain reaction of a photocatalyst agent of the invention;

FIG. 8 plots ultraviolet ray versus eliminated amount of NOₓ;

FIG. 9 plots duration versus eliminated amount of NO for recovering rate while eliminating NOₓ;

FIG. 10 schematically shows a structure of nano-sized metal-oxide and binder;

FIG. 11 plots larger particle and smaller particle versus lowering rate of concentration of methylene blue for comparison purposes;

FIG. 12 is a table showing concentration, duration, and total lowering rate of the invention for eliminating formaldehyde;

FIG. 13 is a bar graph for comparing the efficiencies of 0-3 layers of TiO₂ for treating various bacteria; and

FIG. 14 is a bar graph showing the effect of irradiating light of different wavelengths through a TiO₂ coating for comparing bacteria killing efficiency of them.

### DETAILED DESCRIPTION OF THE INVENTION

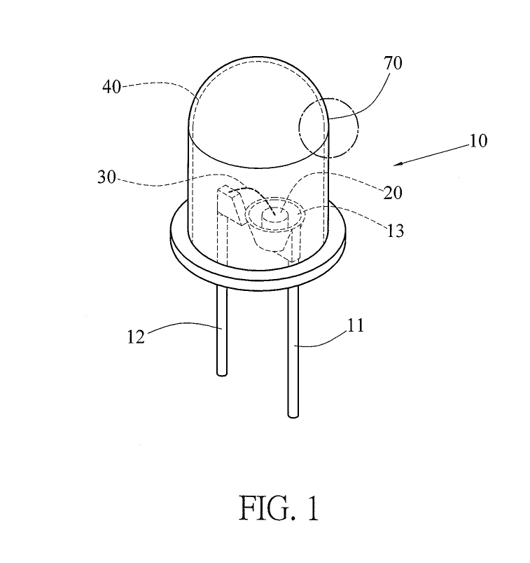

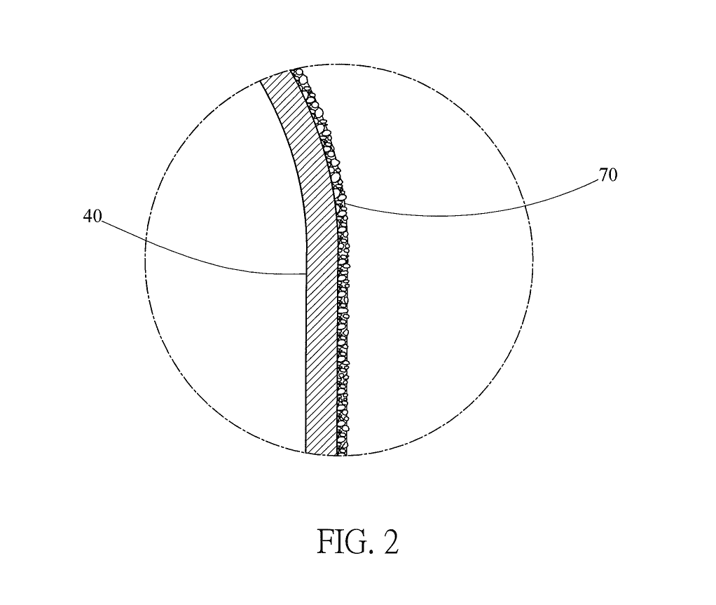

Referring to FIGS. 1 and 2, an LED 10 in accordance with the invention comprises the following components as discussed in detailed below.

An epoxy lens 40 is provided. A cathode lead 11 and an anode lead 12 extend downward from a bottom of the epoxy lens 40. A reflective cup 13 is disposed in the epoxy lens 40. A LED chip 20 is disposed in the epoxy lens 40 and is connected to a bottom of the reflective cup 13 for electrically connecting to the cathode lead 11. Light emitted by the LED chip 20 has a wavelength less than 400 nm. A wire bond 30 is disposed in the epoxy lens 40 and has one end electrically connected to the LED chip 20 and the other end electrically connected to the anode lead 12. A layer of photocatalyst agents 70 is coated on an outer surface of the epoxy resin 40.

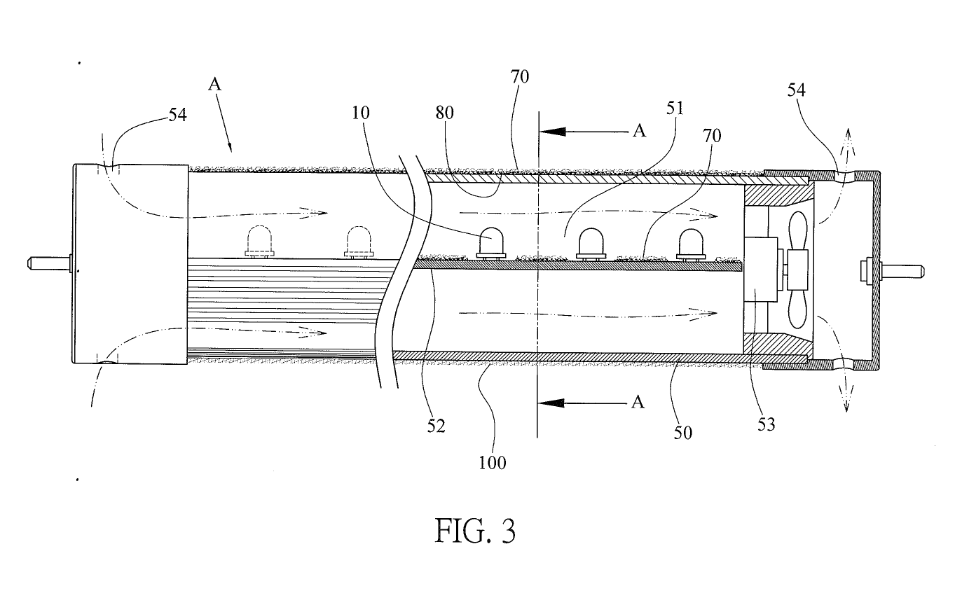

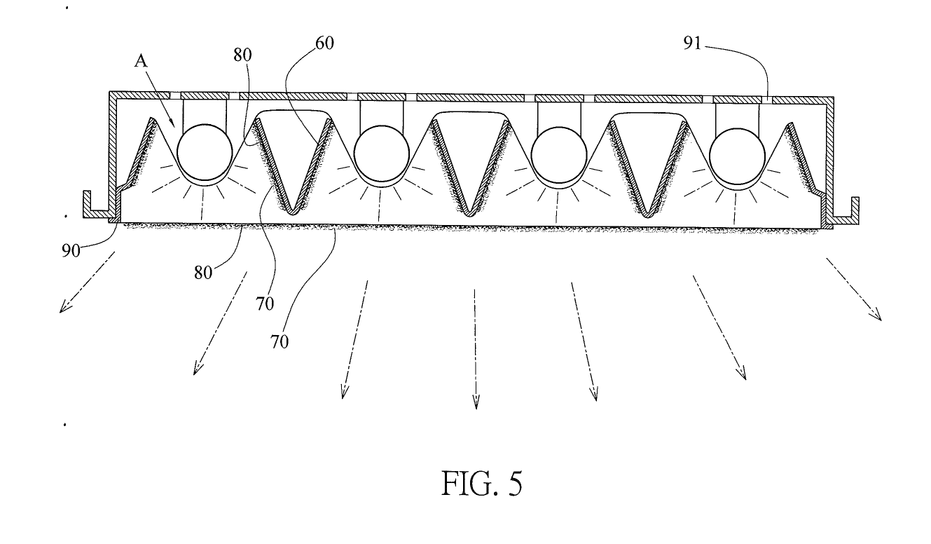

Referring to FIGS. 3 to 5, a light source A incorporating the LEDs 10 as a component of an LED lamp in accordance with a first preferred embodiment is shown. The light source A comprises a hollow cylindrical housing 50 including a space 51 for accommodating a circuit board 52, a plurality of LEDs 10 mounted on the circuit board 52 and electrically connected to the circuit board 52, and a fan 53 electrically connected to the circuit board 52; a plurality of openings 54 formed on the housing 50 communicating with the space 51 so that an activation of the fan 53 can increase ventilation of the space 51 by drawing cold air into the space 51 and expelling hot air out of the space 51 all through the openings 54; a diffusing board 60 provided externally of the housing 50; and a plurality of groups of photocatalyst agents 70 coated on an outer surface of the housing 50 and around the LEDs 10, the groups of photocatalyst agents 70 on the outer surface of the housing 50 being configured to receive light emitted by the LEDs 10.

The groups of photocatalyst agents 70 and the LED chips 20 are separated. The groups of photocatalyst agents 70 include a nano-sized metal-oxide so as to have the capabilities of higher oxidation, stability, and safety. The photocatalyst agents are preferably TiO₂ have a particle size in the range of 50 and 300 nano meters. Ventilation in the space 51 can be increased by activating the fan 53. And in turn, more photocatalyst agents are in contact with air so as to kill more microorganisms in the air. The LED lamp having photocatalyst agents can be installed in an inlet of an air conditioner. It is understood that outlet of an air conditioner has a lower temperature. Dew may be formed at the outlet and it can adversely affect activity of nanoparticles of the photocatalyst agent. To the contrast, inlet of the air conditioner is drier with less dew being formed. It is preferred to install the LED lamp and an additional fan at the inlet of the air conditioner so as to increase ventilation. Therefore, the photocatalyst agents can perform the functions of disinfection, deodorization, and mildew proofing.

A circular layer of fluorescent substance 80 is coated on the outer surface of the housing 50 with the groups of photocatalyst agents 70 formed thereon. An additional layer of fluorescent substance 80 is disposed below the groups of photocatalyst agents 70 around the LEDs 10. The layer of fluorescent substance 80 on the outer surface of the housing 50 is configured to receive light emitted by the LEDs 10. Further, the layer of fluorescent substance 80 can be formed on the groups of photocatalyst agents 70 which in turn are formed on the diffusing board 60. Furthermore, the layer of fluorescent substance 80 can be formed on an outer surface of the groups of photocatalyst agents 70 both on a bottom of a lamp casing 90. In addition, a plurality of openings 91 formed on the lamp casing 90 can increase ventilation of the lamp casing 90.

Light emitted by the LEDs 10 can impinge the fluorescent substance 80 to generate fluorescent light. White light is generated when light emitted by the LEDs 10 mixes with the fluorescent light. The fluorescent substance 80 formed on the housing 50 and the diffusing board 60 is expensive. Thus, it is preferred to use the fluorescent substance 80 can be used again after replacing a malfunctioned LED with a new one. This can reduce the consumption of fluorescent substance. In addition, a layer of nano-silver 100 is formed on a portion of the outer surface of the housing 50 not lit by light emitted by the LEDs 10. The provision of the nano-silver 100 can kill microorganisms in a space not lit by the light emitted by the LEDs 10.

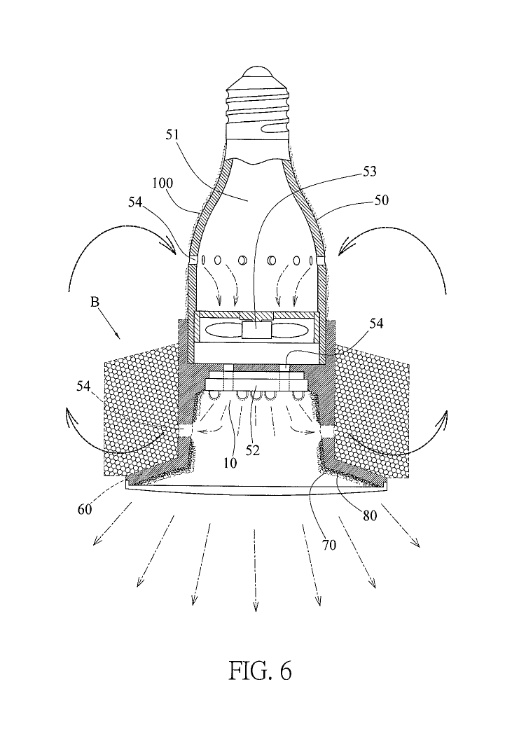

Referring to FIG. 6, a light source B incorporating the LEDs 10 as a component of an LED lamp in accordance with a second preferred embodiment is shown. The light source is shaped a bulb. The characteristics of the second preferred embodiment are described in detail below. The groups of photocatalyst agents 70 are formed on the fluorescent substance 80 which in turn is formed on the diffusing board 60. The nano-silver 100 are formed on an upper portion of the outer surface of the housing 50. The LEDs 10 are exposed on the bottom of the housing 50. The circuit board 52 is provided on top of the LEDs 10 and is disposed in the housing 51.

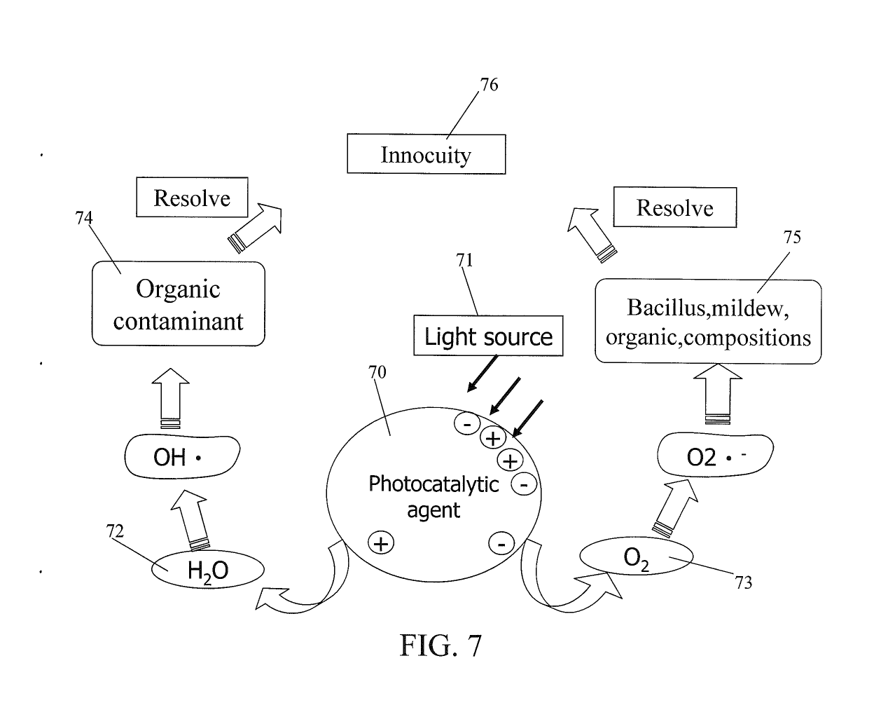

Referring to FIG. 7 in conjunction with FIGS. 1 to 6, the LED chip 20 emits light 71 to the photocatalyst agents 70 on the epoxy lens 40. And in turn, the light impinges the housing 50, the diffusing board 60 and beyond. The light has the functions of disinfection, deodorization, and mildew proofing. The photocatalyst agents 70 can resolve H₂O (hydrogen oxide) 72 to OH— and H+ so that higher oxidation is reacted in order to resolve organic contaminant 74 into innocuity 76. The O₂ 73 is able to resolve organic compositions 75 as bacillus, mildew, etc. into innocuity 76.

Size of an LED is smaller and is more convenient to install than conventional light bulbs. Comparing the invention to a conventional LED of the same size, the invention can increase the total surface area contacting air, so that the functions of disinfection, deodorization, and mildew proofing can be effectively performed.

Customers at public places of entertainment such as KTV, liquor shops, etc. may smoke cigarettes, drink alcohol, etc., and it can produce smell. Using the invention in such places can solve the problem. Moreover, the invention can be in incorporated in a Christmas light to be aesthetic.

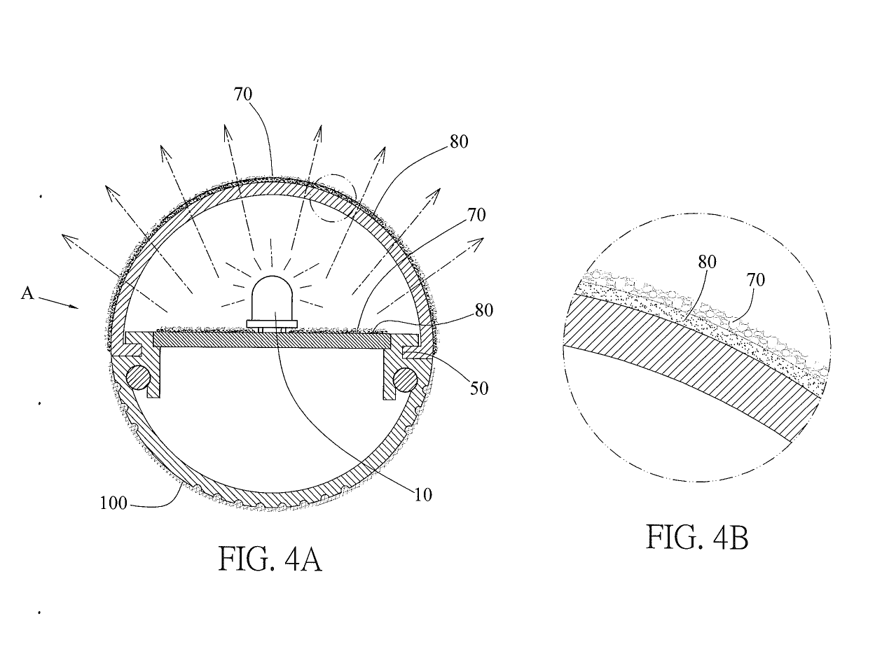

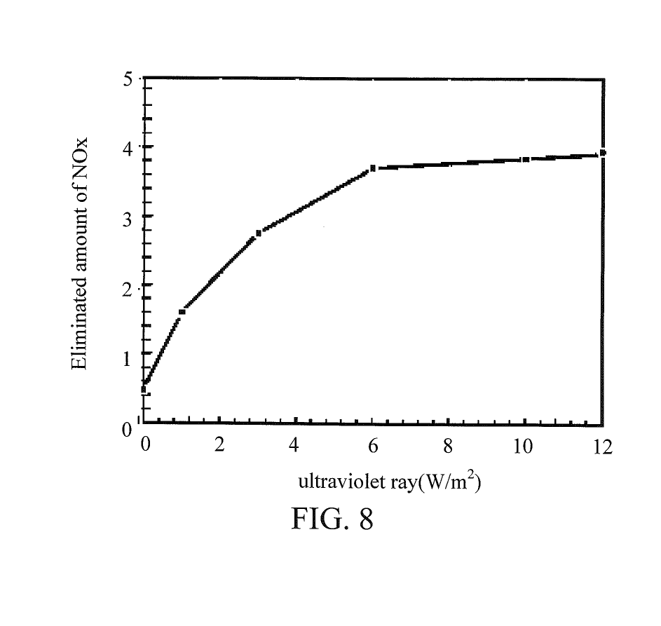

FIG. 8 plots ultraviolet ray versus eliminated amount of NOₓ. According to FIG. 4, the eliminated amount of NOₓ is proportional to the strength of the ultraviolet.

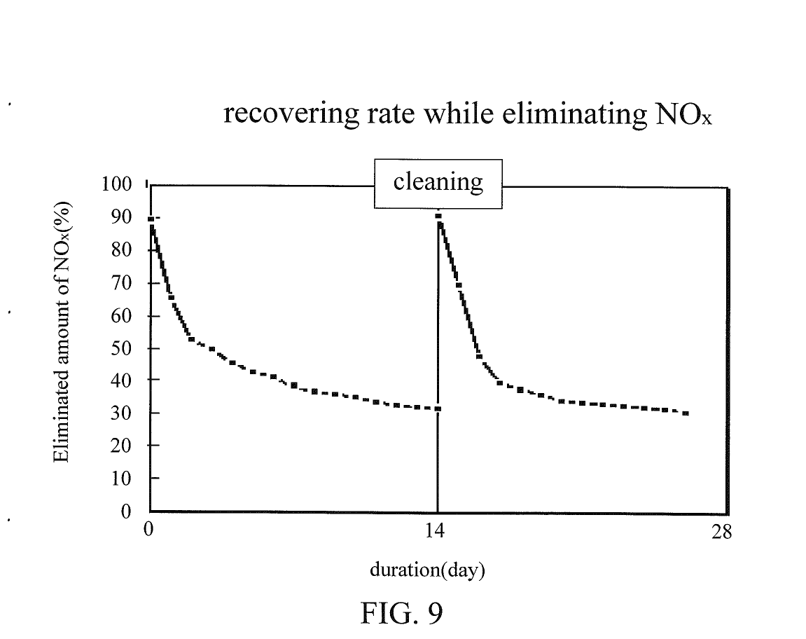

FIG. 9 plots duration versus eliminated amount of NOₓ for recovering rate while eliminating NOₓ. As shown, from day 0 to day 14, the capability to eliminate NOₓ is gradually decreased when continuously using the invention. The capability can be recovered after cleaning the invention, for example, the day after day 14.

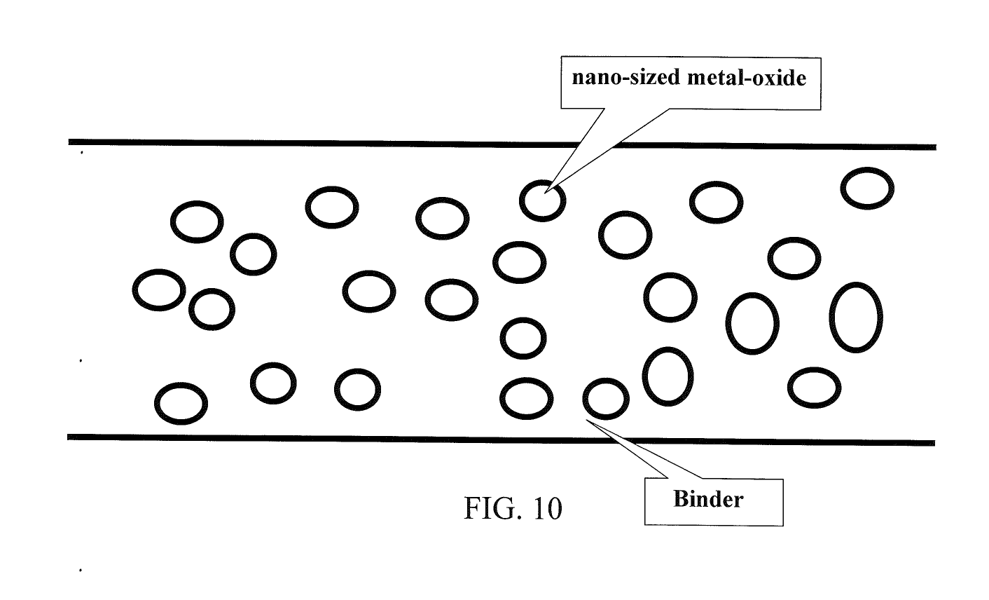

FIG. 10 schematically shows a structure of nano-sized metal-oxide and binder.

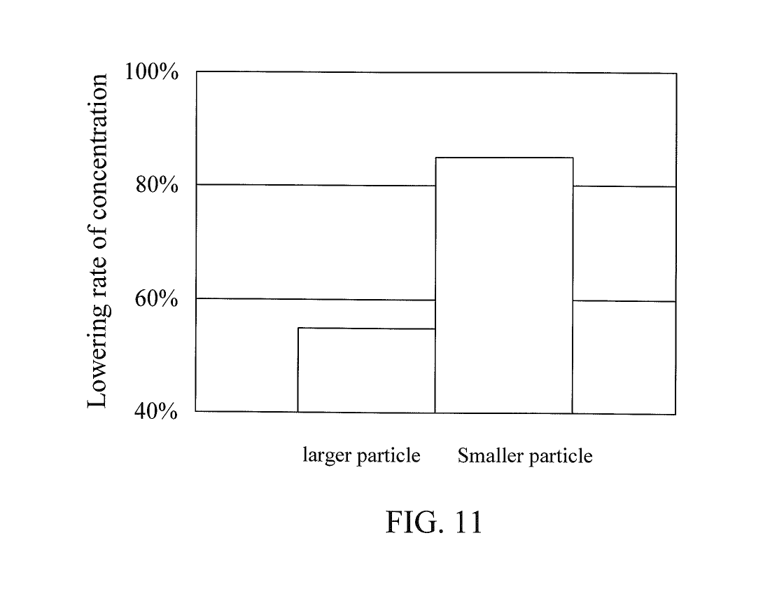

FIG. 11 plots larger particle and smaller particle versus lowering rate of concentration of methylene blue for comparison purposes. As shown, the lowering rate of the concentration with smaller LEDs is higher than the lowering rate of the concentration with larger LEDs. That is, the total curved surface area of the smaller LEDs is larger than that of the larger LEDs.

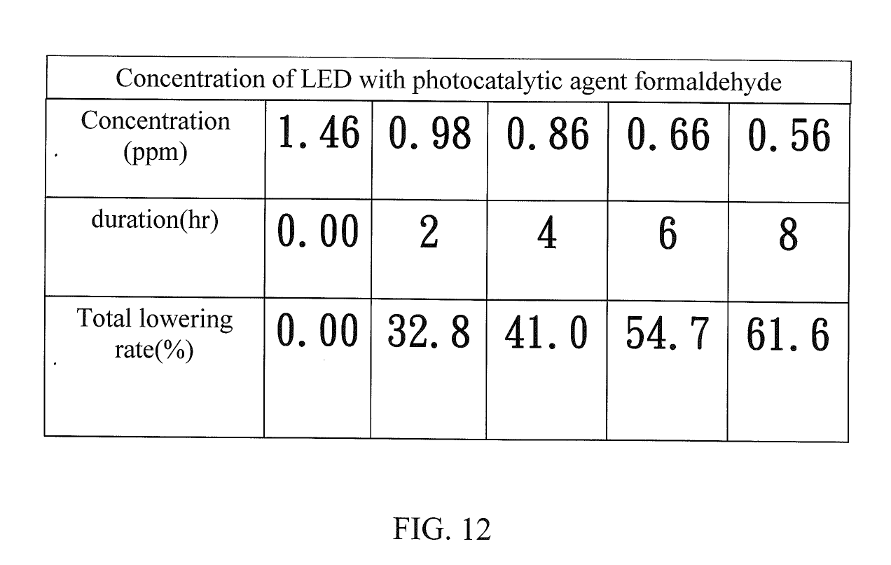

FIG. 12 is a table showing concentration, duration, and total lowering rate of the invention for eliminating formaldehyde. As shown, the total lowering rate is proportional to the duration.

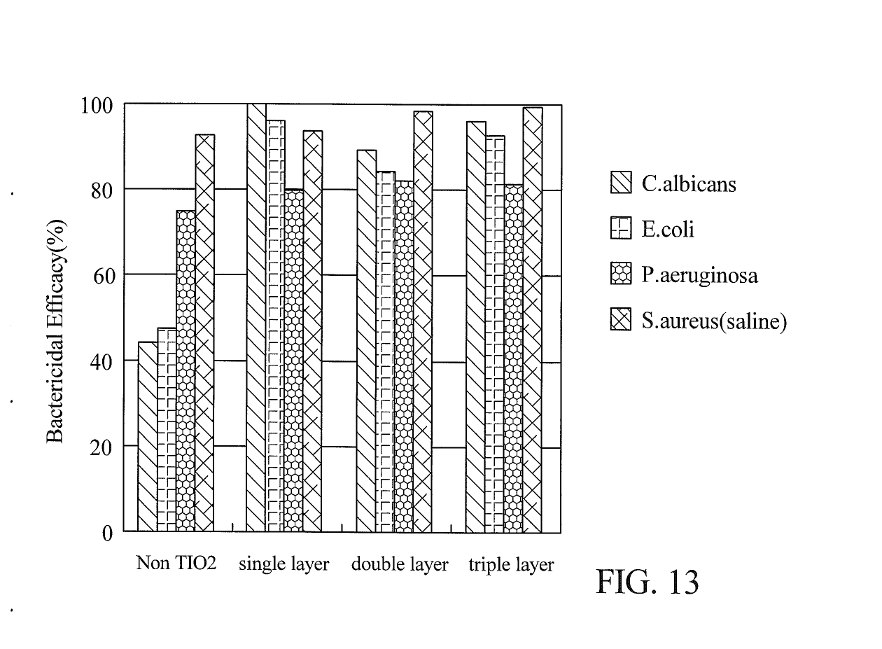

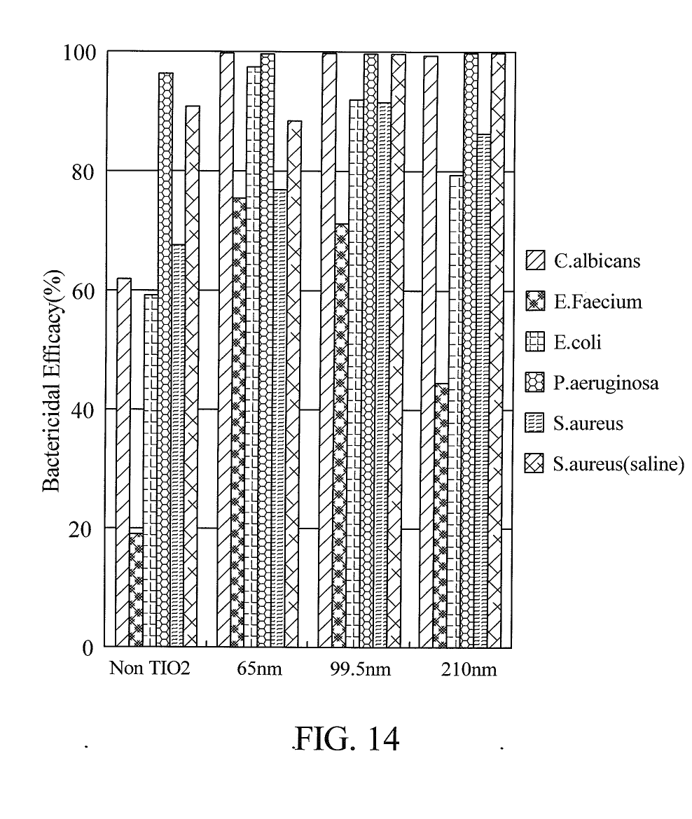

FIG. 13 is a bar graph for comparing the efficiencies of 0-3 layers of TiO₂ for treating various bacteria, and FIG. 14 is a bar graph showing the effect of irradiating light of different wavelengths through a TiO₂ coating for comparing bacteria killing efficiency of them. As shown, the coatings consist of resin, solvent and nano-crystalline TiO₂ which were sprayed or spread on the surfaces and are enabled for self-cleaning, deodorization and disinfection. When the surface of the TiO₂ is irradiated with light having wavelengths less than 385 nm, free radicals are formed, causing organic compounds to decompose. Such a surface, therefore, has the functions of self-cleaning, deodorization and disinfection. Sunlight contains sufficient UV light. In an indoors environment, the blue or UV LEDs also emit light that includes a sufficient fraction having wavelengths less than 385 nm.

The TiO₂-containing coatings were sprayed on glass surfaces, forming thin films when dried. Several pathogenic bacteria were suspended in glycerol solution, and then were smeared on the films and then irradiated with fluorescent light. Results of Li's reference indicate that the nano-crystalline TiO₂ containing coating have substantial bactericidal ability against Escherichia coli BCRC11634, staphylococcus aureus BCRC10451, Pseudomonas aeruginosa BCRC12450, Enterococcus faecium BCRC10067 and Candida albicans BCRC20511.

There is a minor difference between the coatings with different thicknesses (layers). It indicates that the key point of bactericidal efficacy is not the thickness but the surface of the coating. Although the TiO₂ containing coatings are sprayed on the surface with 1, 2 or 3-layer, the surface areas of TiO₂ coating is unchanged and the bactericidal efficacy is the same. As shown in FIG. 14, regarding the nano particle size results, one may conclude that the total bactericidal efficacy of TiO₂ with smaller particle sizes is better. The preferred range is a particle size range is between about 50 and 300 nm.

Reference: Mr. Lien-min Li, “INFLUENCES OF PREPARATION CONDITIONS ON BACTERICIDAL EFFICACY OF TIO2 CONTAINING COATING”, thesis for Master of Science, Department of Bioengineering, Tatung University, TAIWAN, June 2004.

While the invention has been described in terms of preferred embodiments, those skilled in the art will recognize that the invention can be practiced with modifications within the spirit and scope of the appended claims.

What is claimed is:

1. An LED lamp, comprising:
at least one LED chip, a cathode lead extending from a bottom of the LED chip, an anode lead extending from the bottom of the LED chip, and the light emitted by the LED chip has a wavelength less than 400 nm;
a housing for accommodating a circuit board, the at least one LED mounted on the circuit board and electrically connected to the circuit board;
at least one opening formed on the housing for active communicating with inside of the housing;
a plurality of groups of photocatalyst agents coated on areas lit by the LED chip of the at least one LED wherein the groups of photocatalyst agents are separated from the LED chip of the at least one LED, and the photocatalyst agents are TiO₂ having a particle size in the range of 50 and 300 nano meters; and
a plurality of layers of fluorescent substance coated on transparent areas lit by the LED chip wherein light emitted by the LED chip is configured to impinge the layers of fluorescent substance to generate fluorescent light.

2. The LED lamp of claim 1, wherein the housing is either cylindrical or bulb-shaped.

3. The LED lamp of claim 1, further comprising a diffusing board disposed externally of the housing.

4. The LED lamp of claim 3, wherein the groups of photocatalyst agents are coated on an outer surface of the housing or the diffusing board.

5. The LED lamp of claim 3, wherein the layers of fluorescent substance are coated on the housing or the diffusing board.

6. The LED lamp of claim 2, wherein the groups of photocatalyst agents and the layers of fluorescent substance are coated on a lamp casing when the housing is cylindrical.

7. The LED lamp of claim 1, further comprising a layer of nano-silver is coated on the housing.

8. The LED lamp of claim 6, wherein at least one opening is formed on the lamp casing and connecting to a air condition opening to perform active communicating.

9. The LED lamp of claim 1, wherein at least one fan is electrically connected to the circuit board to perform active communicating.
